(19) United States
(12) Patent Application Publication
(10) Pub. No.: US 2020/0345745 A1
(43) Pub. Date: Nov. 5, 2020

(54) KYNURENINE 3-MONOOXYGENASE (KMO) INHIBITORS, AND USES AND COMPOSITIONS THEREOF

(71) Applicants: University of Leicester, Leicester (GB); University of Manchester, Manchester (GB)

(72) Inventors: Nigel Scrutton, Manchester (GB); Flaviano Giorgini, Leicester (GB)

(21) Appl. No.: 16/766,065

(22) PCT Filed: Nov. 20, 2018
(86) PCT No.: PCT/GB2018/053352

§ 371 Date: May 21, 2020

(30) Foreign Application Priority Data
Nov. 21, 2017 (GB) 1719327.7

(51) Int. Cl. A61K 31/538; C07D 265/36; C07D 413/06; A61K 45/06

(52) U.S. Cl. CPC A61K 31/538; A61K 45/06; C07D 413/06; C07D 265/36

(57) ABSTRACT

There is provided compounds of formula (I) or pharmaceutically-acceptable salts thereof, wherein: L, X, R¹, R² and n have meanings provided in the description, which compounds are useful in the treatment of neurodegenerative and neuroinflammatory diseases.

The present invention relates to novel compounds and compositions, and their use in the treatment of neurodegenerative and neuroinflammatory disorders. In particular, the invention relates to novel compounds, compositions and methods for the treatment of neurodegenerative and neuroinflammatory disorders, such as Alzheimer's disease, Parkinson's disease, amyotrophic lateral sclerosis and Huntington's disease, through specific potent inhibition of kynurenine 3-monooxygenase (KMO).

The listing or discussion of an apparently prior-published document in this specification should not necessarily be taken as an acknowledgment that the document is part of the state of the art or is common general knowledge.

In recent years, great interest has been focused on the role of alterations in kynurenine pathway (KP) metabolism in the pathogenesis of several neurodegenerative disorders (Maddison, D. C. & Giorgini, F., Semin. Cell Dev. Biol., 40, 134-141 (2015)).

This metabolic pathway, by which >95% of L-tryptophan is degraded in mammals, produces several neuroactive intermediates which have been found to modulate neurodegeneration and associated phenotypes in models of Alzheimer's disease (AD), Parkinson's disease (PD), amyotrophic lateral sclerosis (ALS) and Huntington's disease (HD) (see: Giorgini, F., et al., Nat. Genet., 37, 526-531 (2005); Giorgini, F., et al., J. Biol. Chem., 283, 7390-7400 (2008); Campesan, S., et al., Curr. Biol., 21, 961-966 (2011); Zwilling, D., et al., Cell, 145, 863-874 (2011); Breda, C., et al., Proc. Natl. Acad. Sci. USA, 113, 5435-5440 (2016); Lee, J. M., et al. Neuropharmacology, 112, 346-364 (2017); Beamont, V., et al., Exp. Neurol., 282, 99-118 (2016)).

Indeed, several studies with patient samples and in model systems suggest that an imbalance in the KP leading to increased levels of the excitotoxin quinolinic acid (QUIN) and the free radical generator 3-hydroxykynurenine (3-HK) relative to the neuroprotective metabolite kynurenic acid (KYNA) contributes to pathogenesis in these disorders (Schwarcz, R., Bruno, J. P., Muchowski, P. J & Wu, H. Q., Nat. Rev. Neurosci., 13, 465-477 (2012)). Notably, upregulation of the central “neurotoxic” branch of the KP is linked to inflammation, with key proinflammatory cytokines such as interferon-IFN-γ directly activating expression of indoleamine-2,3-dioxygenase 1 (IDO1), which acts at the initial step of the KP, cleaving the indole ring of L-tryptophan.

The flavoprotein kynurenine 3-monooxygenase (KMO) lies at a key branchpoint in the pathway regulating flux through either the central neurotoxic branch of the pathway (3-HK and QUIN) or the neuroprotective sidearm forming KYNA. Indeed, inhibition of KMO activity leads to decreased synthesis of downstream metabolites such as 3-HK and QUIN, while increasing levels of KYNA, generating a more “neuroprotective” environment, and making KMO inhibition a promising therapeutic strategy for neurodegenerative disorders (Giorgini, F., et al., J. Biol. Chem., 288, 36554-36566 (2013). KMO requires flavin adenine dinucleotide (FAD) for enzymatic activity and has a six-stranded antiparallel β-sheet domain (Amaral, M., et al., Nature, 496, 382-385 (2013), a structure common to FAD-dependent hydroxylases.

Over the past approximately 20 years, many KMO inhibiting compounds have been developed as experimental tools for modulating KP metabolism in vivo and in vitro (Smith, J. R., Jamie, J. F. & Guillemin, G. J., Drug discovery today, 21, 315-324 (2016)). Close analogues of the KMO substrate L-kynurenine have been found to be effective KMO inhibitors, including the 4-aryl-4-oxobutanoic acid derivatives m-nitrobenzoylalanine (IC50=0.9 μM) (Pellicciari, R., et al., J. Med. Chem, 37, 647-655 (1994)), (R,S)-3,4-dichlorobenzoylalanine (IC50=0.9 μM) (Specialie, C., et al., Eur. J. Pharmacol., 315, 263-267 (1996)) and UPF-648 (IC50=20 nM) (Amori, L., et al., J. Neurochem., 109, 316-325 (2009)). Notably, these compounds can shift KP metabolism towards KYNA synthesis in the brain and are neuroprotective in several disease models (Cozzi, A., Capenedo, R. & Moroni, F., J. Cereb. Blood Flow Metab., 19, 771-777 (1999); Ceresoli-Borroni, G., J. Neurosci. Res., 85, 845-854 (2007)).

The most widely studied KMO inhibitor is Ro-61-8048, a N-(4-phenylthiazol-2-yl) benzenesulfonamide which is a high-affinity inhibitor (IC50=37 nM) of KMO both in vitro and in vivo (Rover, S., et al., J. Med. Chem., 40, 4378-4386 (1997)), and has been used in a number of experimental paradigms for studying the efficacy of KMO inhibition.

The compound JM6, a prodrug which releases Ro 61-8048, improves phenotypes in rodent models of AD, HD, and neuropathic pain (Rojewska, E., et al., Neuropharmacology, 102, 80-91 (2016)), though its mechanism of action has been debated (Beconi, M. G., et al., Drug Metab. Dispos., 40, 2297-2306 (2012)).

Recently, two novel high potency KMO inhibitors have been described—GSK180 and CHDI-340246. GSK180 (IC50=7 μM) is an oxazolidinone, which modulates KP metabolism in vivo and ameliorates disease phenotypes in rodent models of acute pancreatitis (Mole, D. J., et al., Nature medicine, 22, 202-209 (2016)). Oral treatment of CHDI-340246 (IC50=0.5 nM)—a pyrimidine carboxylic acid (Toledo-Sherman, L. M., et al., J. Med. Chem., 58, 1159-1183 (2015))—modules KP metabolism both in the periphery and the CNS, and improves electrophysiological alterations in HD model mice.

Notably, the available KMO inhibitors act predominantly in the periphery by raising levels of L-kynurenine in the blood, which is actively transported across the blood-brain barrier and preferentially converted into KYNA in the CNS. It is likely that brain penetrant KMO inhibitors would be more efficacious for treating neurological disorders than peripherally acting compounds, as inhibition of KMO in the CNS leads to increased KYNA levels and decreased levels of 3-HK and QUIN.

Nonetheless, despite the many efforts to develop KMO inhibitors for use in the clinic, to date no compounds that robustly penetrate the blood-brain barrier are available.

It has now been found that certain benzoxamines have surprising properties which render such compounds useful in the treatment of neurodegenerative and neuroinflammatory disorders, such as Alzheimer's disease, Parkinson's disease, amyotrophic lateral sclerosis, and Huntington's disease. In particular, it is thought that these compounds are able to penetrate the blood-brain barrier, thus rendering them particularly suitable for use as part of a medical treatment.

### Compounds of the Invention

In a first aspect of the invention, there is provided a compound of formula I

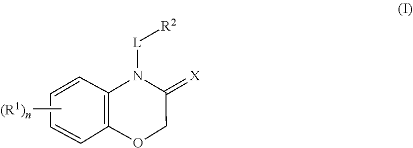

or a pharmaceutically-acceptable salt thereof, wherein:
L represents C₁₋₆ alkyl optionally substituted by one or more fluoro;
n represents 0 to 4;
each R¹ independently represents halo, Ra¹, —CN, -A^{a1}-C(Q^{a1})R^{b1}, -A^{b1}-X(Q^{b1})N(R^{c1})R^{d1}, -A^{c1}-C(Q^{c1})OR^{e1}, -A^{d1}-S(O)ₚR^{f1}, -A^{e1}-S(O)ₚN(R^{g1})R^{h1}, -A^{f1}-S(O)ₚORⁱ¹, —N₃, —N(R^{j1})R^{k1}, —N(H)CN, —NO₂, —ONO₂, —OR^{l1} or —SR^{m1};
each A^{a1} to A^{f1} independently represents a single bond, —N(R^{p1})— or —O—;
each Q^{a1} to Q^{c1} independently represents ═O, ═S, ═NRⁿ¹ or ═N(OR^{o1});
each R^{a1} and R^{f1} independently represents C₁₋₆ alkyl optionally substituted by one or more groups independently selected from G^{1a}, or heterocycloalkyl optionally substituted by one or more groups independently selected from G^{1b};
each R^{p1} independently represents H or C₁₋₆ alkyl optionally substituted by one or more fluoro;
each R^{b1}, R^{c1}, R^{d1}, R^{e1}, R^{g1}, R^{h1}, Rⁱ¹, R^{j1}, R^{k1}, R^{l1}, R^{m1}, Rⁿ¹ and R^{o1} independently represents H, C₁₋₆ alkyl optionally substituted by one or more groups independently selected from G^{1a} or heterocycloalkyl optionally substituted by one or more groups independently selected from G^{1b}, or
alternatively any of R^{c1} and R^{d1}, R^{g1} and R^{h1} and/or R^{j1} and R^{k1} may be linked together to form, together with the nitrogen atom to which they are attached, a 3- to 6-membered ring, which ring optionally contains one further heteroatom and which ring optionally is substituted by one or more groups independently selected from halo, C₁₋₃ alkyl optionally substituted by one or more halo, and =0;
each G^{1a} and G^{1b} independently represents halo, —CN, —N(Ra³)Rb³, —OR^{c3}, —SR^{d3} or ═O;
each R^{a3}, R^{b3}, R^{c3} and R^{d3} independently represents H or C₁₋₆ alkyl optionally substituted by one or more fluoro;
or R^{a3} and R^{b3} are linked together to form, along with the nitrogen atom to which they are attached, a 3- to 6-membered ring, which ring optionally contains one further heteroatom and which ring optionally is substituted by one or more groups independently selected from fluoro, C₁₋₃ alkyl optionally substituted by one or more fluoro, and ═O;
each p and q independently represents 1 or 2;
R² represents —C(O)OR^{a2}, —C(O)NR^{b2}R^{c2}, or tetrazole;
each R^{a2}, R^{b2} and R^{c2} independently represents H, C₁₋₆ alkyl, phenyl, or CH₂-phenyl, wherein the latter three groups may be optionally substituted by one or more fluoro; and
X represents O or S,
which compounds may be referred to herein as compounds of the invention.

In a particular embodiment of the first aspect, in the compounds of the invention of formula (I), or a pharmaceutically acceptable salt thereof:

n represents 2 to 4;
each R¹ independently represents C₁₋₆ alkyl group optionally substituted by one or more groups independently selected from G^{1a}, wherein at least one R¹ is a C₁₋₆ alkyl group optionally substituted by one or more groups independently selected from G^{1a}.

For example, the compounds of the invention, or a pharmaceutically acceptable salt thereof, may have at least two R¹ groups which are a C₁₋₆ alkyl group optionally substituted by one or more groups independently selected from G^{1a}, such as two R¹ groups which are methyl.

The skilled person will understand that references herein to compounds of the invention will include references to all embodiments and particular forms thereof. In some aspects, “compounds of the invention” include 2-(6-chloro-5,7-dimethyl-3-oxo-1,4-benzoxazin-4-yl)acetic acid and 3-(6-chloro-3-oxo-2,3-dihydro-4H-1,4-benzoxazin-4-yl)propanoic acid.

Unless indicated otherwise, all technical and scientific terms used herein have the same meaning as commonly understood by one of ordinary skill in the art to which this invention pertains.

Pharmaceutically-acceptable salts include acid addition salts and base addition salts. Such salts may be formed by conventional means, for example by reaction of a free acid or a free base form of a compound of the invention with one or more equivalents of an appropriate acid or base, optionally in a solvent, or in a medium in which the salt is insoluble, followed by removal of said solvent, or said medium, using standard techniques (e.g. in vacuo, by freeze-drying or by filtration). Salts may also be prepared by exchanging a counter-ion of a compound of the invention in the form of a salt with another counter-ion, for example using a suitable ion exchange resin.

Particular acid addition salts that may be mentioned include carboxylate salts (e.g. formate, acetate, trifluoroacetate, propionate, isobutyrate, heptanoate, decanoate, caprate, caprylate, stearate, acrylate, caproate, propiolate, ascorbate, citrate, glucuronate, glutamate, glycolate, α-hydroxybutyrate, lactate, tartrate, phenylacetate, mandelate, phenylpropionate, phenylbutyrate, benzoate, chlorobenzoate, methyl benzoate, hydroxybenzoate, methoxybenzoate, dinitrobenzoate, o-acetoxybenzoate, salicylate, nicotinate, isonicotinate, cinnamate, oxalate, malonate, succinate, suberate, sebacate, fumarate, malate, maleate, hydroxymaleate, hippurate, phthalate or terephthalate salts), halide salts (e.g. chloride, bromide or iodide salts), sulphonate salts (e.g. benzenesulphonate, methyl-, bromo- or chloro-benzenesulphonate, xylenesulphonate, methanesulphonate, ethanesulphonate, propanesulphonate, hydroxyethanesulphonate, 1- or 2-naphthalene-sulphonate or 1,5-naphthalenedisulphonate salts) or sulphate, pyrosulphate, bisulphate, sulphite, bisulphite, phosphate, monohydrogenphosphate, dihydrogenphosphate, metaphosphate, pyrophosphate or nitrate salts, and the like.

Particular base addition salts that may be mentioned include salts formed with alkali metals (such as Na and K salts), alkaline earth metals (such as Mg and Ca salts), organic bases (such as ethanolamine, diethanolamine, triethanolamine, tromethamine and lysine) and inorganic bases (such as ammonia and aluminium hydroxide). More particularly, base addition salts that may be mentioned include Mg, Ca and, most particularly, K and Na salts.

For the avoidance of doubt, compounds of the invention may exist as solids, and thus the scope of the invention includes all amorphous, crystalline and part crystalline forms thereof, and may also exist as oils. Where compounds of the invention exist in crystalline and part crystalline forms, such forms may include solvates, which are included in the scope of the invention. Compounds of the invention may also exist in solution.

Compounds of the invention may contain double bonds and may thus exist as E (entgegen) and Z (zusammen) geometric isomers about each individual double bond. All such isomers and mixtures thereof are included within the scope of the invention.

Compounds of the invention may also exhibit tautomerism. All tautomeric forms and mixtures thereof are included within the scope of the invention.

Compounds of the invention may also contain one or more asymmetric carbon atoms and may therefore exhibit optical and/or diastereoisomerism. Diastereoisomers may be separated using conventional techniques, e.g. chromatography or fractional crystallisation. The various stereoisomers may be isolated by separation of a racemic or other mixture of the compounds using conventional, e.g. fractional crystallisation or HPLC, techniques. Alternatively the desired optical isomers may be made by reaction of the appropriate optically active starting materials under conditions which will not cause racemisation or epimerisation (i.e. a ‘chiral pool’ method), by reaction of the appropriate starting material with a ‘chiral auxiliary’ which can subsequently be removed at a suitable stage, by derivatisation (i.e. a resolution, including a dynamic resolution); for example, with a homochiral acid followed by separation of the diastereomeric derivatives by conventional means such as chromatography, or by reaction with an appropriate chiral reagent or chiral catalyst all under conditions known to the skilled person. All stereoisomers and mixtures thereof are included within the scope of the invention.

As used herein, references to halo and/or halogen will independently refer to fluoro, chloro, bromo and iodo (for example, fluoro and chloro).

Unless otherwise specified, C_{1-z} alkyl groups (where z is the upper limit of the range) defined herein may be straight-chain or, when there is a sufficient number (i.e. a minimum of two or three, as appropriate) of carbon atoms, be branched-chain, and/or cyclic (so forming a C_{3-z}-cycloalkyl group). When there is a sufficient number (i.e. a minimum of four) of carbon atoms, such groups may also be part cyclic. Part cyclic alkyl groups that may be mentioned include cyclopropylmethyl and cyclohexylethyl. When there is a sufficient number of carbon atoms, such groups may also be multicyclic (e.g. bicyclic or tricyclic) or spirocyclic. Such alkyl groups may also be saturated or, when there is a sufficient number (i.e. a minimum of two) of carbon atoms, be unsaturated (forming, for example, a C₂-z alkenyl or a C_{2-z} alkynyl group).

Unless otherwise specified, C_{1-z} alkylene groups (where z is the upper limit of the range) defined herein may (in a similar manner to the definition of C_{1-z} alkyl) be straight-chain or, when there is a sufficient number (i.e. a minimum of two or three, as appropriate) of carbon atoms, be branched-chain, and/or cyclic (so forming a C₃₋ₜ-cycloalkylene group). When there is a sufficient number (i.e. a minimum of four) of carbon atoms, such groups may also be part cyclic. When there is a sufficient number of carbon atoms, such groups may also be multicyclic (e.g. bicyclic or tricyclic) or spirocyclic. Such alkylene groups may also be saturated or, when there is a sufficient number (i.e. a minimum of two) of carbon atoms, be unsaturated (forming, for example, a C_{2-z} alkenylene or a C_{2-z} alkynylene group). Particular alkylene groups that may be mentioned include those that are straight-chained or cyclic and saturated.

As used herein, the term heterocycloalkyl may refer to non-aromatic monocyclic and bicyclic heterocycloalkyl groups (which groups may further be bridged) in which at least one (e.g. one to four) of the atoms in the ring system is other than carbon (i.e. a heteroatom), and in which the total number of atoms in the ring system is between three and twelve (e.g. between five and ten and, most preferably, between three and eight, e.g. a 5- or 6-membered heterocycloalkyl group). Further, such heterocycloalkyl groups may be saturated or unsaturated containing one or more double and/or triple bonds, forming for example a C_{2-z} (e.g. C_{4-z}) heterocycloalkenyl (where z is the upper limit of the range) or a C_{7-z} heterocycloalkynyl group. C_{2-z} heterocycloalkyl groups that may be mentioned include 7-azabicyclo-[2.2.1]heptanyl, 6-azabicyclo[3.1.1]heptanyl, 6-azabicyclo[3.2.1]-octanyl, 8-azabicyclo[3.2.1]octanyl, aziridinyl, azetidinyl, 2,3-dihydroisothiazolyl, dihydropyranyl, dihydropyridyl, dihydropyrrolyl (including 2,5-dihydropyrrolyl), dioxolanyl (including 1,3-dioxolanyl), dioxanyl (including 1,3-dioxanyl and 1,4-dioxanyl), dithianyl (including 1,4-dithianyl), dithiolanyl (including 1,3-dithiolanyl), imidazolidinyl, imidazolinyl, isothiazolidinyl, morpholinyl, 7-oxabicyclo[2.2.1]heptanyl, 6-oxabicyclo[3.2.1]-octanyl, oxetanyl, oxiranyl, piperazinyl, piperidinyl, pyranyl, pyrazolidinyl, pyrrolidinonyl, pyrrolidinyl, pyrrolinyl, quinuclidinyl, sulpholanyl, 3-sulpholenyl, tetrahydropyranyl, tetrahydrofuryl, tetrahydropyridyl (such as 1,2,3,4-tetrahydropyridyl and 1,2,3,6-tetrahydropyridyl), thietanyl, thiiranyl, thiolanyl, tetrahydrothiopyranyl, thiomorpholinyl, trithianyl (including 1,3,5-trithianyl), tropanyl and the like. Substituents on heterocycloalkyl groups may, where appropriate, be located on any atom in the ring system including a heteroatom. Further, in the case where the substituent is another cyclic compound, then the cyclic compound may be attached through a single atom on the heterocycloalkyl group, forming a so-called “spiro”-compound. The point of attachment of heterocycloalkyl groups may be via any atom in the ring system including (where appropriate) a further heteroatom (such as a nitrogen atom), or an atom on any fused carbocyclic ring that may be present as part of the ring system. Heterocycloalkyl groups may also be in the N- or S-oxidised form.

At each occurrence when mentioned herein, particular heterocycloalkyl groups that may be mentioned include 3- to 8-membered heterocycloalkyl groups (e.g. a 4- to 6-membered heterocycloalkyl group).

As may be used herein, the term aryl includes references to C₆₋₁₄ (e.g. C₆₋₁₀) aromatic groups. Such groups may be monocyclic or bicyclic and, when bicyclic, be either wholly or partly aromatic. C₆₋₁₀ aryl groups that may be mentioned include phenyl, naphthyl, 1,2,3,4-tetrahydronaphthyl, indanyl, and the like (e.g. phenyl, naphthyl and the like, such as phenyl). For the avoidance of doubt, the point of attachment of substituents on aryl groups may be via any carbon atom of the ring system.

As may be used herein, the term heteroaryl (or heteroaromatic) includes references to 5- to 14- (e.g. 5- to 10-) membered heteroaromatic groups containing one or more heteroatoms selected from oxygen, nitrogen and/or sulphur. Such heteroaryl groups may comprise one, two, or three rings, of which at least one is aromatic. Substituents on heteroaryl/heteroaromatic groups may, where appropriate, be located on any atom in the ring system including a heteroatom. The point of attachment of heteroaryl/heteroaromatic groups may be via any atom in the ring system including (where appropriate) a heteroatom. Bicyclic heteroaryl/heteroaromatic groups may comprise a benzene ring fused to one or more further aromatic or non-aromatic heterocyclic rings, in which instances, the point of attachment of the polycyclic heteroaryl/heteroaromatic group may be via any ring including the benzene ring or the heteroaryl/heteroaromatic or heterocycloalkyl ring. Examples of heteroaryl/heteroaromatic groups that may be mentioned include pyridinyl, pyrrolyl, furanyl, thiophenyl, oxadiazolyl, thiadiazolyl, thiazolyl, oxazolyl, pyrazolyl, triazolyl, tetrazolyl, isoxazolyl, isothiazolyl, imidazolyl, imidazopyrimidinyl, imidazothiazolyl, thienothiophenyl, pyrimidinyl, furopyridinyl, indolyl, azaindolyl, pyrazinyl, pyrazolopyrimidinyl, indazolyl, pyrimidinyl, quinolinyl, isoquinolinyl, quinazolinyl, benzofuranyl, benzothiophenyl, benzoimidazolyl, benzoxazolyl, benzothiazolyl, benzotriazolyl and purinyl. The oxides of heteroaryl/heteroaromatic groups are also embraced within the scope of the invention (e.g. the N-oxide). As stated above, heteroaryl includes polycyclic (e.g. bicyclic) groups in which one ring is aromatic (and the other may or may not be aromatic). Hence, other heteroaryl groups that may be mentioned include e.g. benzo[1,3]dioxolyl, benzo[1,4]dioxinyl, dihydrobenzo[d]isothiazole, 3,4-dihydrobenz[1,4]oxazinyl, dihydrobenzothiophenyl, indolinyl, 5H, 6H, 7H-pyrrolo[1,2-b]pyrimidinyl, 1,2,3,4-tetrahydroquinolinyl, thiochromanyl and the like.

For the avoidance of doubt, as used herein, references to heteroatoms will take their normal meaning as understood by one skilled in the art. Particular heteroatoms that may be mentioned include phosphorus, selenium, tellurium, silicon, boron, oxygen, nitrogen and sulphur (e.g. oxygen, nitrogen and sulphur).

For the avoidance of doubt, references to polycyclic (e.g. bicyclic) groups (e.g. when employed in the context of heterocycloalkyl groups) will refer to ring systems wherein more than two scissions would be required to convert such rings into a straight chain, with the minimum number of such scissions corresponding to the number of rings defined (e.g. the term bicyclic may indicate that a minimum of two scissions would be required to convert the rings into a straight chain). For the avoidance of doubt, the term bicyclic (e.g. when employed in the context of heterocycloalkyl groups) may refer to groups in which the second ring of a two-ring system is formed between two adjacent atoms of the first ring, and may also refer to groups in which two non-adjacent atoms are linked by either an alkylene or heteroalkylene chain (as appropriate), which later groups may be referred to as bridged.

For the avoidance of doubt, when an aryl or an heteroaryl group is substituted with a group via a double bond, such as ═O, it is understood that the aryl or heteroaryl group is partly aromatic, i.e. the aryl or heteroaryl group consists of at least two rings where at least one ring is not aromatic.

The present invention also embraces isotopically-labelled compounds of the present invention which are identical to those recited herein, but for the fact that one or more atoms are replaced by an atom having an atomic mass or mass number different from the atomic mass or mass number usually found in nature (or the most abundant one found in nature). All isotopes of any particular atom or element as specified herein are contemplated within the scope of the compounds of the invention. Hence, the compounds of the invention also include deuterated compounds, i.e. in which one or more hydrogen atoms are replaced by the hydrogen isotope deuterium.

For the avoidance of doubt, in cases in which the identity of two or more substituents in a compound of the invention may be the same, the actual identities of the respective substituents are not in any way interdependent. For example, in the situation in which two or more R¹ groups are present, those R¹ groups may be the same or different. Similarly, where two or more R¹ groups are present and each represent R^{a1}, the R^{a1} groups in question may be the same or different. Likewise, when more than one R^{a1} is present and each independently represents C₁₋₆ alkyl substituted by one or more G^{1a} group, the identities of each G^{1a} are in no way interdependent.

For the avoidance of doubt, when a term such as “A^{a1} to A^{f1}” is employed herein, this will be understood by the skilled person to mean A^{a1}, A^{b1}, A^{c1}, A^{d1}, A^{e1} and A^{f1} inclusively. Unless otherwise stated, the same reasoning will apply to other such terms used herein.

The skilled person will appreciate that compounds of the invention that are the subject of this invention include those that are stable. That is, compounds of the invention include those that are sufficiently robust to survive isolation, e.g. from a reaction mixture, to a useful degree of purity.

All embodiments of the invention and particular features mentioned herein may be taken in isolation or in combination with any other embodiments and/or particular features mentioned herein (hence describing more particular embodiments and particular features as disclosed herein) without departing from the disclosure of the invention.

In a particular embodiment of the first aspect of the invention, there is the proviso that the compound of formula I is not:

(1) 2-(6-chloro-5,7-dimethyl-3-oxo-1,4-benzoxazin-4-yl)acetic acid.

Particular compounds of formula I (i.e. compounds of the invention) that may be mentioned include those in which each R¹ independently represents H, halo, R^{a1}, —N(R^{j1})R^{k1} or —OR^{l1}. For example, particular compounds of the invention that may be mentioned include those in which each R¹ independently represents C₁₋₆ alkyl group optionally substituted by one or more groups independently selected from G^{1a} or a halo group, wherein at least one R¹ is a C₁₋₆ alkyl group optionally substituted by one or more groups independently selected from G^{1a}.

Particular compounds of formula I that may be mentioned include those in which each R^{a1} independently represents C₁₋₆ alkyl optionally substituted by one or more fluoro; and each R^{j1}, R^{k1} and R^{l1} independently represents H or C₁₋₆ alkyl optionally substituted by one or more fluoro.

Particular compounds of formula I that may be mentioned include those in which each R^{a2}, R^{b2} and R^{c2} independently represents H or C₁₋₃ alkyl optionally substituted by one or more fluoro.

Particular compounds of formula I that may be mentioned include those in which X represents O.

Particular compounds of formula I that may be mentioned include those in which L represents C₁₋₃ alkyl optionally substituted by one or more fluoro. For example, L represents C₁ alkyl (i.e. methyl) optionally substituted by one or more fluoro.

In a particular embodiment, the compound of formula I is:

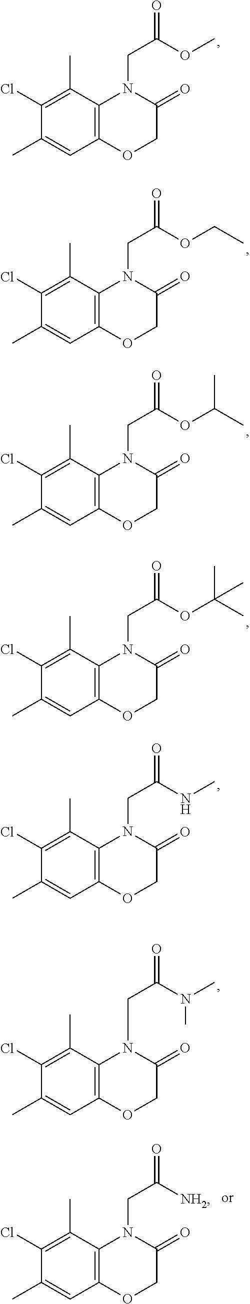

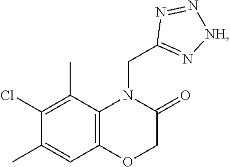 [SMILES: Cc1cc2c(c(C)c1Cl)N(Cc1nn[nH]n1)C(=O)CO2]

or a pharmaceutically-acceptable salt thereof.

### Compositions and Medical Uses

As discussed hereinbefore, compounds of the invention, and therefore compositions and kits comprising the same, are useful as pharmaceuticals.

According to a second aspect of the invention, there is provided a compound of the invention, as hereinbefore defined (i.e. in the first aspect of the invention, including all embodiments and particular features therein), or 2-(6-chloro-5,7-dimethyl-3-oxo-1,4-benzoxazin-4-yl)acetic acid or 3-(6-chloro-3-oxo-2,3-dihydro-4H-1,4-benzoxazin-4-yl)propanoic acid, for use as a pharmaceutical. Further, there is provided a compound of the invention, as hereinbefore defined, or 2-(6-chloro-5,7-dimethyl-3-oxo-1,4-benzoxazin-4-yl)acetic acid or 3-(6-chloro-3-oxo-2,3-dihydro-4H-1,4-benzoxazin-4-yl)propanoic acid for use in medicine.

In a particular embodiment of the second aspect of the invention, there is the proviso that the compound of formula I is not:
(1) 2-(6-chloro-5,7-dimethyl-3-oxo-1,4-benzoxazin-4-yl)acetic acid.

As indicated herein, compounds of the invention (including 2-(6-chloro-5,7-dimethyl-3-oxo-1,4-benzoxazin-4-yl)acetic acid and 3-(6-chloro-3-oxo-2,3-dihydro-4H-1,4-benzoxazin-4-yl)propanoic acid) may be of particular use in the treatment or prophylaxis of neurodegenerative and neuroinflammatory diseases.

Thus, in a third aspect of the invention, there is provided a compound of the invention, as hereinbefore defined (i.e. in the first aspect of the invention, including all embodiments and particular features therein), for use in the treatment of a neurodegenerative or neuroinflammatory disease.

In an alternative third aspect of the invention, there is provided the use of a compound of the invention, as hereinbefore defined, in the manufacture of a medicament for the treatment of a neurodegenerative or neuroinflammatory disease.

In a further alternative third aspect of the invention, there is provided a method of treating a neurodegenerative or neuroinflammatory disease comprising administering to a patient in need thereof a therapeutically effective amount of a compound of the invention or 2-(6-chloro-5,7-dimethyl-3-oxo-1,4-benzoxazin-4-yl)acetic acid or 3-(6-chloro-3-oxo-2,3-dihydro-4H-1,4-benzoxazin-4-yl)propanoic acid.

In a particular embodiment of the third aspect of the invention, there is the proviso that the compound of formula I is not:

(1) 2-(6-chloro-5,7-dimethyl-3-oxo-1,4-benzoxazin-4-yl)acetic acid.)

The skilled person will understand that references to the treatment of a particular condition (or, similarly, to treating that condition) take their normal meanings in the field of medicine. In particular, the terms may refer to achieving a reduction in, a delay of the onset of, or a slowing of the progression (i.e. worsening) of, the severity of one or more clinical symptoms associated with the conditions which may be a reduction in the likelihood of occurrence of such symptoms. For example, in the case of neurodegenerative or neuroinflammatory disease, such as Alzeimer's disease, Parkinson's disease, amyotrophic lateral sclerosis and Huntington's disease, the term may refer to achieving a reduction in the severity of the associated neurological symptom.

As used herein, the term prophylaxis will include references to the prevention (and, similarly, preventing) of the disease or disorder (and vice-versa). As such, references to prophylaxis may also be references to prevention, and vice versa. In particular, such terms term may refer to achieving a reduction (for example, at least a 10% reduction, such as at least a 20%, 30% or 40% reduction, e.g. at least a 50% reduction) in the likelihood of the patient (or healthy subject) developing the condition (which may be understood as meaning that the condition of the patient changes such that patient is diagnosed by a physician as having, e.g. requiring treatment for, the relevant disease or disorder).

As used herein, references to patients will refer to a living subject being treated, including mammalian (e.g. human) patients.

As used herein, the term effective amount will refer to an amount of a compound that confers a therapeutic effect on the treated patient. The effect may be objective (i.e. measurable by some test or marker) or subjective (i.e. the subject gives an indication of and/or feels an effect).

Although compounds of the invention (including 2-(6-chloro-5,7-dimethyl-3-oxo-1,4-benzoxazin-4-yl)acetic acid and 3-(6-chloro-3-oxo-2,3-dihydro-4H-1,4-benzoxazin-4-yl)propanoic acid) may possess pharmacological activity as such, certain pharmaceutically-acceptable (e.g. “protected”) derivatives of these compounds may exist or be prepared which may not possess such activity, but may be administered parenterally or orally and thereafter be metabolised in the body to form these compounds. Such compounds (which may possess some pharmacological activity, provided that such activity is appreciably lower than that of the active compounds to which they are metabolised) may therefore be described as “prodrugs” of these compounds.

As used herein, references to prodrugs will include compounds that form a compound of the invention, in an experimentally-detectable amount, within a predetermined time, following enteral or parenteral administration (e.g. oral or parenteral administration). All prodrugs of the compounds of the invention (including 2-(6-chloro-5,7-dimethyl-3-oxo-1,4-benzoxazin-4-yl)acetic acid and 3-(6-chloro-3-oxo-2,3-dihydro-4H-1,4-benzoxazin-4-yl)propanoic acid) are included within the scope of the invention.

Furthermore, certain compounds of the invention may possess no or minimal pharmacological activity as such, but may be administered parenterally or orally, and thereafter be metabolised in the body to form compounds of the invention that possess pharmacological activity as such. Such compounds (which also includes compounds that may possess some pharmacological activity, but that activity is appreciably lower than that of the active compounds of the invention to which they are metabolised), may also be described as “prodrugs”.

Thus, the compounds of the invention (including 2-(6-chloro-5,7-dimethyl-3-oxo-1,4-benzoxazin-4-yl)acetic acid and 3-(6-chloro-3-oxo-2,3-dihydro-4H-1,4-benzoxazin-4-yl)propanoic acid) are useful because they possess pharmacological activity, and/or are metabolised in the body following oral or parenteral administration to form compounds that possess pharmacological activity.

As indicated herein, the compounds of the invention (including 2-(6-chloro-5,7-dimethyl-3-oxo-1,4-benzoxazin-4-yl)acetic acid and 3-(6-chloro-3-oxo-2,3-dihydro-4H-1,4-benzoxazin-4-yl)propanoic acid) may be useful in the treatment of a neurodegenerative or neuroinflammatory disease.

Particular neurodegenerative or neuroinflammatory diseases that may be mentioned include those selected from the group consisting of: Alzheimer's disease, Parkinson's disease, amyotrophic lateral sclerosis and Huntington's disease.

The skilled person will understand that treatment with compounds of the invention (including 2-(6-chloro-5,7-dimethyl-3-oxo-1,4-benzoxazin-4-yl)acetic acid and 3-(6-chloro-3-oxo-2,3-dihydro-4H-1,4-benzoxazin-4-yl)propanoic acid) may further comprise (i.e. be combined with) further treatment(s) for the same condition. In particular, treatment with compounds of the invention may be combined with means for the treatment of neurodegenerative and neuroinflammatory diseases, such as treatment with one or more other therapeutic agent that is useful in the in the treatment of neurodegenerative and neuroinflammatory diseases and/or one or more physical method used in the treatment of neurodegenerative and neuroinflammatory diseases (such as treatment through surgery), as known to those skilled in the art.

Compounds of the invention (including 2-(6-chloro-5,7-dimethyl-3-oxo-1,4-benzoxazin-4-yl)acetic acid and 3-(6-chloro-3-oxo-2,3-dihydro-4H-1,4-benzoxazin-4-yl)propanoic acid) will normally be administered orally, intravenously, subcutaneously, buccally, rectally, dermally, nasally, tracheally, bronchially, sublingually, intranasally, topically, by any other parenteral route or via inhalation, in a pharmaceutically-acceptable dosage form.

Compounds of the invention (including 2-(6-chloro-5,7-dimethyl-3-oxo-1,4-benzoxazin-4-yl)acetic acid and 3-(6-chloro-3-oxo-2,3-dihydro-4H-1,4-benzoxazin-4-yl)propanoic acid) may be administered alone or may be administered by way of known pharmaceutical compositions/formulations, including tablets, capsules or elixirs for oral administration, suppositories for rectal administration, sterile solutions or suspensions for parenteral or intramuscular administration, and the like.

According to a fourth aspect of the invention there is thus provided a pharmaceutical composition/formulation comprising a compound of the invention, as hereinbefore defined (i.e. in the first aspect of the invention) or 2-(6-chloro-5,7-dimethyl-3-oxo-1,4-benzoxazin-4-yl)acetic acid or 3-(6-chloro-3-oxo-2,3-dihydro-4H-1,4-benzoxazin-4-yl)propanoic acid, and optionally (e.g. in admixture with) one or more pharmaceutically-acceptable adjuvant, diluent and/or carrier.

In a particular embodiment of the fourth aspect of the invention, there is the proviso that the compound of formula I is not a compound selected from the list consisting of:

(1) 2-(6-chloro-5,7-dimethyl-3-oxo-1,4-benzoxazin-4-yl)acetic acid.

The skilled person will understand that references herein to compounds of the invention (including 2-(6-chloro-5,7-dimethyl-3-oxo-1,4-benzoxazin-4-yl)acetic acid and 3-(6-chloro-3-oxo-2,3-dihydro-4H-1,4-benzoxazin-4-yl)propanoic acid) being for particular uses (and, similarly, to uses and methods of use relating to these compounds) may also apply to pharmaceutical compositions comprising these compounds.

Compounds of the invention (including 2-(6-chloro-5,7-dimethyl-3-oxo-1,4-benzoxazin-4-yl)acetic acid and 3-(6-chloro-3-oxo-2,3-dihydro-4H-1,4-benzoxazin-4-yl)propanoic acid) may be administered in the form of tablets or capsules, e.g. time-release capsules that are taken orally. Alternatively, the compounds may be in a liquid form and may be taken orally or by injection. The compounds may also be in the form of suppositories, or, creams, gels, and foams e.g. that can be applied to the skin. In addition, they may be in the form of an inhalant that is applied nasally or via the lungs.

The skilled person will understand that compounds may act systemically and/or locally (i.e. at a particular site).

Compounds of the invention (including 2-(6-chloro-5,7-dimethyl-3-oxo-1,4-benzoxazin-4-yl)acetic acid and 3-(6-chloro-3-oxo-2,3-dihydro-4H-1,4-benzoxazin-4-yl)propanoic acid) may be administered orally, intravenously, subcutaneously, buccally, rectally, dermally, nasally, tracheally, bronchially, by any other parenteral route or via inhalation, in a pharmaceutically-acceptable dosage form. Alternatively, particularly where the compounds are intended to act locally, compounds of they may be administered topically.

Thus, in a particular embodiment, the pharmaceutical formulation is provided in a pharmaceutically-acceptable dosage form, including tablets or capsules, liquid forms to be taken orally or by injection, suppositories, creams, gels, foams, or inhalants (e.g. to be applied intranasally). For the avoidance of doubt, in such embodiments, compounds of the invention (including 2-(6-chloro-5,7-dimethyl-3-oxo-1,4-benzoxazin-4-yl)acetic acid and 3-(6-chloro-3-oxo-2,3-dihydro-4H-1,4-benzoxazin-4-yl)propanoic acid) may be present as a solid (e.g. a solid dispersion), liquid (e.g. in solution) or in other forms, such as in the form of micelles.

In a more particular embodiment, the pharmaceutical formulation is provided in the form of tablets or capsules, liquid forms to be taken orally or by injection (e.g. a form suitable for intravenous injection). In particular, injection may take place using conventional means, and may include the use of microneedles.

Depending on e.g. potency and physical characteristics of the compound of the invention (including 2-(6-chloro-5,7-dimethyl-3-oxo-1,4-benzoxazin-4-yl)acetic acid and 3-(6-chloro-3-oxo-2,3-dihydro-4H-1,4-benzoxazin-4-yl)propanoic acid) (i.e. active ingredient), pharmaceutical formulations that may be mentioned include those in which the active ingredient is present in at least 1% (or at least 10%, at least 30% or at least 50%) by weight. That is, the ratio of active ingredient to the other components (i.e. the addition of adjuvant, diluent and carrier) of the pharmaceutical composition is at least 1:99 (or at least 10:90, at least 30:70 or at least 50:50) by weight.

As described herein, compounds of the invention (including 2-(6-chloro-5,7-dimethyl-3-oxo-1,4-benzoxazin-4-yl)acetic acid and 3-(6-chloro-3-oxo-2,3-dihydro-4H-1,4-benzoxazin-4-yl)propanoic acid) may also be combined with one or more other (i.e. different, e.g. agents other than compounds of formula I) therapeutic agents that are useful in the treatment of neurodegenerative or neuroinflammatory diseases. Such combination products that provide for the administration of a compound of the invention, 2-(6-chloro-5,7-dimethyl-3-oxo-1,4-benzoxazin-4-yl)acetic acid or 3-(6-chloro-3-oxo-2,3-dihydro-4H-1,4-benzoxazin-4-yl)propanoic acid in conjunction with one or more other therapeutic agent may be presented either as separate formulations, wherein at least one of those formulations comprises a compound of the invention, 2-(6-chloro-5,7-dimethyl-3-oxo-1,4-benzoxazin-4-yl)acetic acid or 3-(6-chloro-3-oxo-2,3-dihydro-4H-1,4-benzoxazin-4-yl)propanoic acid, and at least one comprises the other therapeutic agent, or may be presented (i.e. formulated) as a combined preparation (i.e. presented as a single formulation including a compound of the invention, 2-(6-chloro-5,7-dimethyl-3-oxo-1,4-benzoxazin-4-yl)acetic acid or 3-(6-chloro-3-oxo-2,3-dihydro-4H-1,4-benzoxazin-4-yl)propanoic acid and the one or more other therapeutic agent).

Thus, according to a fifth aspect of the invention, there is provided a combination product comprising:

(A) a compound of the invention, as hereinbefore defined (i.e. in the first aspect of the invention) or 2-(6-chloro-5,7-dimethyl-3-oxo-1,4-benzoxazin-4-yl)acetic acid or 3-(6-chloro-3-oxo-2,3-dihydro-4H-1,4-benzoxazin-4-yl)propanoic acid; and
(B) one or more other therapeutic agent that is useful in the treatment of a neurodegenerative or neuroinflammatory disease, wherein each of components (A) and (B) is formulated in admixture, optionally with one or more a pharmaceutically-acceptable adjuvant, diluent or carrier.

In a sixth aspect of the invention there is provided a kit-of-parts comprising:

(a) a pharmaceutical formulation as hereinbefore defined; and
(b) one or more other therapeutic agent that is useful in the treatment of cancer, optionally in admixture with one or more pharmaceutically-acceptable adjuvant, diluent or carrier,
which components (a) and (b) are each provided in a form that is suitable for administration in conjunction (i.e. concomitantly or sequentially) with the other.

In a particular embodiment of the fifth and sixth aspects of the invention, there is the proviso that the compound of formula I is not:

(1) 2-(6-chloro-5,7-dimethyl-3-oxo-1,4-benzoxazin-4-yl)acetic acid.

Compounds of the invention, 2-(6-chloro-5,7-dimethyl-3-oxo-1,4-benzoxazin-4-yl)acetic acid or 3-(6-chloro-3-oxo-2,3-dihydro-4H-1,4-benzoxazin-4-yl)propanoic acid may be administered at varying doses. Oral, pulmonary and topical dosages (and subcutaneous dosages, although these dosages may be relatively lower) may range from about 0.01 mg/kg of body weight per day (mg/kg/day) to about 100 mg/kg/day, preferably about 0.01 to about 10 mg/kg/day, and more preferably about 0.1 to about 5.0 mg/kg/day. For e.g. oral administration, the compositions typically contain from about 0.01 mg to about 2000 mg, for example from about 0.1 mg to about 500 mg, or from 1 mg to about 100 mg, of the active ingredient. Intravenously, the most preferred doses will range from about 0.001 to about 10 mg/kg/hour during constant rate infusion. Advantageously, compounds may be administered in a single daily dose, or the total daily dosage may be administered in divided doses of two, three or four times daily.

In any event, the physician, or the skilled person, will be able to determine the actual dosage which will be most suitable for an individual patient, which is likely to vary with the route of administration, the type and severity of the condition that is to be treated, as well as the species, age, weight, sex, renal function, hepatic function and response of the particular patient to be treated. The above-mentioned dosages are exemplary of the average case; there can, of course, be individual instances where higher or lower dosage ranges are merited, and such are within the scope of this invention.

### Preparation of Compounds/Compositions

Pharmaceutical compositions/formulations, combination products and kits as described herein may be prepared in accordance with standard and/or accepted pharmaceutical practice.

Thus, in a further aspect of the invention there is provided a process for the preparation of a pharmaceutical composition/formulation, as hereinbefore defined, which process comprises bringing into association a compound of the invention, as hereinbefore defined, or 2-(6-chloro-5,7-dimethyl-3-oxo-1,4-benzoxazin-4-yl)acetic acid or 3-(6-chloro-3-oxo-2,3-dihydro-4H-1,4-benzoxazin-4-yl)propanoic acid with one or more pharmaceutically-acceptable adjuvant, diluent or carrier.

In further aspects of the invention, there is provided a process for the preparation of a combination product or kit-of-parts as hereinbefore defined, which process comprises bringing into association a compound of the invention, as hereinbefore defined, 2-(6-chloro-5,7-dimethyl-3-oxo-1,4-benzoxazin-4-yl)acetic acid or 3-(6-chloro-3-oxo-2,3-dihydro-4H-1,4-benzoxazin-4-yl)propanoic acid or a pharmaceutically-acceptable salt thereof with the other therapeutic agent that is useful in the treatment of a neurodegenerative or neuroinflammatory disease, and at least one pharmaceutically-acceptable adjuvant, diluent or carrier.

As used herein, references to bringing into association will mean that the two components are rendered suitable for administration in conjunction with each other.

Thus, in relation to the process for the preparation of a kit of parts as hereinbefore defined, by bringing the two components “into association with” each other, we include that the two components of the kit of parts may be:

(i) provided as separate formulations (i.e. independently of one another), which are subsequently brought together for use in conjunction with each other in combination therapy; or
(ii) packaged and presented together as separate components of a “combination pack” for use in conjunction with each other in combination therapy.

Compounds of the invention as described herein may be prepared in accordance with techniques that are well known to those skilled in the art, such as those described in the examples provided hereinafter.

According to a seventh aspect of the invention there is provided a process for the preparation of a compound of the invention as hereinbefore defined, which process comprises:
(i) for compounds of formula I wherein R² represents tetrazole, reaction of a compound of formula II

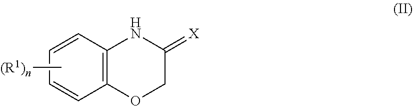

wherein R¹, n and X are as defined herein in formula I (or any particular feature or embodiment thereof), with a compound of formula III

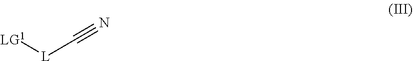

wherein L is as defined herein in formula I (or any particular feature or embodiments thereof) and LG¹ represents a suitable leaving group (such as halo, e.g. bromo), in the presence of a suitable base (such as a weak base, e.g. a weak non-nucleophilic base, such as an alkali metal carbonate, e.g. K₂CO₃) and in the presence of a suitable solvent (such as a polar organic solvent, e.g. N,N′-dimethyacetamide, N,N′-dimethylformamide or tetrahydrofuran), under conditions known to those skilled in the art, followed by
reaction of the resultant compound of formula IV

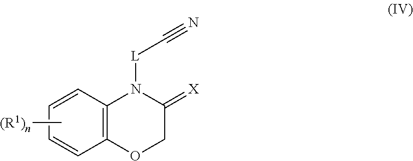

wherein R¹, n, X and L are as defined herein in formula I (or any particular feature or embodiments thereof), with an azide (such as an alkali metal azide, for example sodium azide) in the presence of a suitable co-reagent (such as iodine, silica-supported sodium hydrogen sulphate, triethylammonium chloride, zinc chloride, aluminium trichloride, or ammonium chloride) and in the presence of a suitable solvent (such as a polar organic solvent, e.g. N,N′-dimethyformamide, methanol, propanol or butanol), under conditions known to those skilled in the art; or
(ii) for compounds of formula I wherein R² represents C(O)OR^{a2} and R^{a2} does not represent H, reaction of a compound of formula II

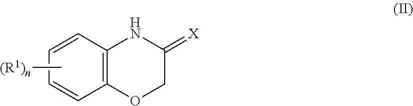

wherein R¹ and X are as defined herein in formula I (or any particular feature or embodiment thereof), with a compound of formula V

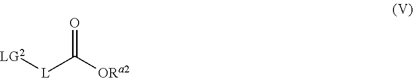

wherein L and R^{a2} are as defined herein in formula I (or any particular feature or embodiments thereof) and LG² represents a suitable leaving group (such as halo, e.g. bromo), in the presence of a suitable base (e.g. such as a strong base, e.g. sodium hydride) and in the presence of a suitable solvent (such as a polar organic solvent, e.g. N,N′-dimethyacetamide, N,N′-dimethylformamide or tetrahydrofuran), under conditions known to those skilled in the art; or
(iii) for compounds of formula I wherein R² represents C(O)OR^{a2} and R^{a2} represents H, reaction of a compound of formula IA

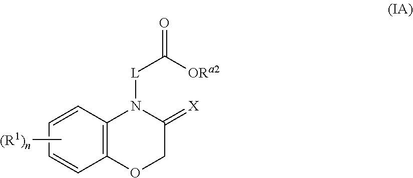

wherein R¹, n, R^{a2}, X and L are as defined herein in formula I (or any particular feature of embodiments thereof), with a suitable aqueous base (such as lithium hydroxide monohydrate) in the presence of a suitable solvent (such as a polar organic solvent, e.g. N,N′-dimethyacetamide, N,N′-dimethylformamide or tetrahydrofuran), under conditions known to those skilled in the art; or
(iv) for compounds of formula I wherein R² represents C(O)NR^{b2}R^{c2}, reaction of a compound of formula IB

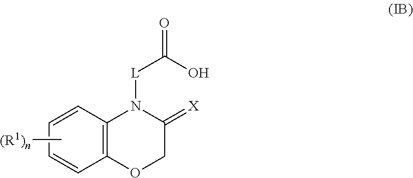

wherein R¹, n, X and L are as defined herein in formula I (or any particular feature of embodiments thereof), with a compound of formula VI

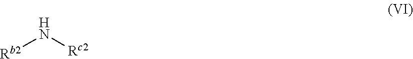

wherein R^{b2} and R^{c2} are as defined herein formula I (or any particular or feature of embodiments thereof), in the presence of a suitable coupling reagent (such as 1,1′-carbonyldiimidazole, N,N′ dicyclohexylcarbodiimide or N-(3-dimethylaminopropyl)-N′-ethyl carbodiimide) and in the presence of a suitable solvent (such as dichloromethane), optionally in the presence of a suitable base (such as a sterically hindered base, e.g. 3-dimethylaminopyridine, triethylamine or N,N-diisopropylethlamine), under conditions known to those skilled in the art.

Compounds of formulae II, III, V and VI are either commercially available, are known in the literature, or may be obtained either by analogy with the processes described herein, or by conventional synthetic procedures, in accordance with standard techniques, from available starting materials using appropriate reagents and reaction conditions. In this respect, the skilled person may refer to inter alia “Comprehensive Organic Synthesis” by B. M. Trost and I. Fleming, Pergamon Press, 1991. Further references that may be employed include “Heterocyclic Chemistry” by J. A. Joule, K. Mills and G. F. Smith, 3rd edition, published by Chapman & Hall, “Comprehensive Heterocyclic Chemistry II” by A. R. Katritzky, C. W. Rees and E. F. V. Scriven, Pergamon Press, 1996 and “Science of Synthesis”, Volumes 9-17 (Hetarenes and Related Ring Systems), Georg Thieme Verlag, 2006.

Compounds of the invention may be isolated from their reaction mixtures and, if necessary, purified using conventional techniques as known to those skilled in the art. Thus, processes for preparation of compounds of the invention as described herein may include, as a final step, isolation and optionally purification of the compound of the invention (e.g. isolation and optionally purification of the compound of formula I).

It will be appreciated by those skilled in the art that, in the processes described above and hereinafter, the functional groups of intermediate compounds may need to be protected by protecting groups. The protection and deprotection of functional groups may take place before or after a reaction in the above-mentioned schemes.

Protecting groups may be applied and removed in accordance with techniques that are well known to those skilled in the art and as described hereinafter. For example, protected compounds/intermediates described herein may be converted chemically to unprotected compounds using standard deprotection techniques. The type of chemistry involved will dictate the need, and type, of protecting groups as well as the sequence for accomplishing the synthesis. The use of protecting groups is fully described in “Protective Groups in Organic Synthesis”, 3rd edition, T. W. Greene & P. G. M. Wutz, Wiley-Interscience (1999).

Compounds of the invention (including 2-(6-chloro-5,7-dimethyl-3-oxo-1,4-benzoxazin-4-yl)acetic acid and 3-(6-chloro-3-oxo-2,3-dihydro-4H-1,4-benzoxazin-4-yl)propanoic acid) may have the advantage that they may be more efficacious than, be less toxic than, be longer acting than, be more potent than, produce fewer side effects than, be more easily absorbed than, and/or have a better pharmacokinetic profile (e.g. higher oral bioavailability and/or lower clearance) than, and/or have other useful pharmacological, physical, or chemical properties over, compounds known in the prior art, whether for use in the above-stated indications or otherwise. In particular, compounds of the invention (including 2-(6-chloro-5,7-dimethyl-3-oxo-1,4-benzoxazin-4-yl)acetic acid and 3-(6-chloro-3-oxo-2,3-dihydro-4H-1,4-benzoxazin-4-yl)propanoic acid) may have the advantage that they are more efficacious and/or exhibit advantageous properties in vivo.

Without wishing to be bound by theory, it is believed that compounds of the invention (including 2-(6-chloro-5,7-dimethyl-3-oxo-1,4-benzoxazin-4-yl)acetic acid and 3-(6-chloro-3-oxo-2,3-dihydro-4H-1,4-benzoxazin-4-yl)propanoic acid) will be particularly useful in the treatment of neurodegenerative or neuroinflammatory diseases due to its ability to inhibit KMO in the CNS. This decreases levels of neurotoxic KP metabolites (e.g. 3-HK, QUIN), while increasing levels of the neuroprotective metabolite KYNA. Current compounds are not brain penetrant, and thus can only increase levels of KYNA (via increased levels of kynurenine in the blood, which is transported into the CNS and preferentially converted to KYNA), while not affecting levels of the neurotoxic metabolites. Thus, the compounds of the invention (including 2-(6-chloro-5,7-dimethyl-3-oxo-1,4-benzoxazin-4-yl)acetic acid and 3-(6-chloro-3-oxo-2,3-dihydro-4H-1,4-benzoxazin-4-yl)propanoic acid) are more efficacious based upon the combination of effects described above.

### BRIEF DESCRIPTION OF THE DRAWINGS

FIG. 1 shows results obtained from the experiment described in Biological Example 3 for the compound of Example 5.

FIG. 2 shows results obtained from the experiment described in Biological Example 3 for the compound of Example 10.

FIG. 3 shows results obtained from the experiment described in Biological Example 5 for the compound of Example 5 where the concentration of NADPH was varied.

FIG. 4 shows results obtained from the experiment described in Biological Example 5 for the compound of Example 5 where the concentration of L-Kyn was varied.

FIG. 5 shows the results obtained from the experiment described in Biological Example 6 for the compounds of Example 1 to 3 and 5.

### EXAMPLE COMPOUNDS

The invention is illustrated by way of the followed examples, in which the following abbreviations may be employed.

CDI 1,1′-carbonyldiimidazole
DMF N,N′-dimethylformamide
DTT 1,4-dithiothreitol
FAD flavin adenine dinucleotide
HEPES 4-(2-hydroxyethyl)piperazine-1-ethanesulfonic acid
HPLC high performance liquid chromatography
MS mass spectrometry
MTBE methyl tert-butyl ether
NADPH nicotinamide adenine dinucleotide phosphate
NMR nuclear magnetic resonance
THF tetrahydrofuran
UPLC ultra performance liquid chromatography

Compounds of the invention, and intermediate compounds used in their preparation, were synthesised according to the following procedures.

### Intermediate 1: 4-chloro-3,5-dimethyl-2-nitro-phenol

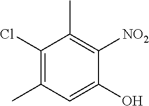

To a solution of 4-chloro-3,5-dimethyl-phenol (30 g, 192 mmol) in acetic acid (450 mL) was added HNO₃ (18.57 g, 192 mmol, 65% purity) at 5° C. The resulting mixture was stirred at 25° C. for 1 h. The reaction mixture was poured into ice-water (500 g) and stirred for 10 min. The resulting solid was collected by filtration, washed with water (200 mL×2) and dried under high vacuum at 50° C. for 24 h to yield 4-chloro-3,5-dimethyl-2-nitro-phenol (36 g, 179 mmol, 93.2% yield) as an orange solid.

¹H NMR (400 MHz, CDCl₃): δ 9.54 (s, 1H), 6.92 (s, 1H), 2.61 (s, 3H), 2.40 (s, 3H).

### Intermediate 2: Methyl 2-(4-chloro-3,5-dimethyl,2-nitro-phenoxy)acetate

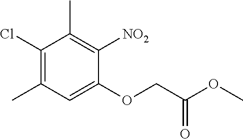

To a solution of the compound of Intermediate 1 (36 g, 179 mmol) and methyl 2-bromoacetate (32.78 g, 214 mmol) in DMF (150 mL) was added K₂CO₃ (37.02 g, 268 mmol) at 25° C. The resulting mixture was heated at 80° C. and stirred for 2 h, before being cooled and poured into water (500 mL). The mixture was stirred for 10 min, filtered, and the resulting solid collected, washed with water (200 mL×2), and dried under high vacuum at 50° C. for 24 h. Methyl 2-(4-chloro-3,5-dimethyl-2-nitro-phenoxy)acetate (40 g, 140 mmol, 78.5% yield) was obtained as a yellow solid of 95.9% purity by HPLC.

¹H NMR (400 MHz, CDCl₃): δ 6.68 (s, 1H), 4.68 (s, 2H), 3.79 (s, 3H), 2.39 (s, 3H), 2.32 (s, 3H).

### Intermediate 3: 6-chloro-5,7-dimethyl-4H-1,4-benzoxazin-3-one

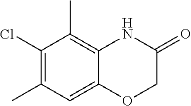

To a solution of the compound of Intermediate 2 (40 g, 146 mmol) in acetic acid (500 mL) was added portionwise iron (81.62 g, 1.46 mol) at 80° C. over 30 min. The mixture was heated to 110° C. and stirred for a further 30 min. The reaction mixture was filtered through celite, and the filter cake washed with hot acetic acid (250 mL×2). The filtrate was concentrated in vacuo, diluted with water (500 mL) and stirred for 10 min. The resulting precipitate was collected by filtration, washed with water (250 mL×2), and dried under high vacuum at 50° C. for 48 h. 6-chloro-5,7-dimethyl-4H-1,4-benzoxazin-3-one (26 g, 123 mmol, 84.1% yield) was obtained as a white solid.

LCMS m/z (M+H)+=(Calculated for C₁₀H₁₀ClNO₂, 212.04) found, 212.2

### Example 1: methyl 2-(6-chloro-5,7-dimethyl-3-oxo-1,4-benzoxazin-4-yl)acetate

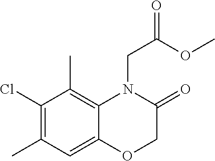

To a solution of the compound of Intermediate 3 (10 g, 47.3 mmol) in DMF (70 mL) was added NaH (3.78 g, 94.5 mmol, 60% purity) at 25° C., and the resulting mixture stirred at 25° C. for 30 min. Methyl 2-bromoacetate (14.46 g, 94.5 mmol) was added dropwise at 25° C., and the mixture stirred at 50° C. for 12 h. The mixture was poured into cooled water (150 mL) and the aqueous extracted with ethyl acetate (50 mL×3). The combined organic layer was washed with saturated aqueous citric acid (80 mL) and brine (50 mL). The organic layer was dried over Na₂SO₄, filtered and concentrated under reduced pressure to give the crude product. The crude product was washed with MTBE (20 mL), and filtered to obtain methyl 2-(6-chloro-5,7-dimethyl-3-oxo-1,4-benzoxazin-4-yl)acetate (8 g, 28.2 mmol, 59.7% yield) as an off-white solid.

¹H NMR (400 MHz, CDCl₃): δ 6.85 (s, 1H), 4.50 (s, 2H), 4.47 (s, 2H), 3.76 (s, 3H), 2.36 (s, 3H), 2.33 (s, 3H);

LCMS: m/z (M+H)⁺=(Calculated for C₁₃H₁₄ClNO₄, 284.06) found, 284.0.

### Example 2: ethyl 2-(6-chloro-5,7-dimethyl-3-oxo-1,4-benzoxazin-4-yl)acetate

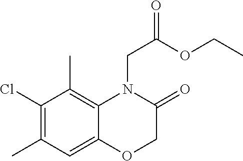 [SMILES: CCOC(=O)CN1C(=O)COc2cc(C)c(Cl)c(C)c21]

To a solution of the compound of Intermediate 3 (500 mg, 2.36 mmol) in DMF (3.5 mL) was added NaH (189 mg, 4.72 mmol, 60% purity) at 25° C., and the resulting mixture stirred at 25° C. for 30 min. Ethyl 2-bromoacetate (789 mg, 4.72 mmol) was added at 25° C., and the mixture stirred at 50° C. for 12 h. The mixture was poured into cooled water (15 mL) and the aqueous extracted with EtOAc (30 mL×2). The combined organic layer was washed with saturated aqueous citric acid (30 mL) and brine (20 mL). The organic layer was dried over Na₂SO₄, filtered and concentrated under reduced pressure to give the crude product. The crude product was purified by prep-HPLC (neutral conditions; column: Agela Durashell C18 150*25 5 u; mobile phase [water(10 mM NH₄CO₃)-ACN]; B %: 42%-72%, 10.5 min) to obtain ethyl 2-(6-chloro-5,7-dimethyl-3-oxo-1,4-benzoxazin-4-yl)acetate (108 mg, 362 μmol, 15.3% yield, 99.7% purity) as an off-white solid.

¹H NMR (400 MHz, CDCl₃): δ 6.85 (s, 1H), 4.50 (s, 2H), 4.46 (s, 2H), 4.21 (q, 2H), 2.36 (s, 3H), 2.33 (s, 3H), 1.26 (t, 3H)′

LCMS: m/z (M+H)+=(Calculated for C₁₄H₁₆ClNO₄, 298.08) found, 298.1

### Example 3: isopropyl 2-(6-chloro-5,7-dimethyl-3-oxo-1,4-benzoxazin-4-yl)acetate

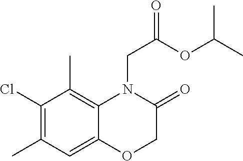 [SMILES: Cc1cc2c(c(C)c1Cl)N(CC(=O)OC(C)C)C(=O)CO2]

To a solution of the compound of Intermediate 3 (500 mg, 2.36 mmol) in DMF (3.5 mL) was added NaH (189 mg, 4.72 mmol, 60% purity) at 25° C., and the resulting mixture stirred at 25° C. for 30 min. Isopropyl 2-bromoacetate (855 mg, 4.72 mmol) was added at 25° C., and the mixture stirred at 50° C. for 12 h. The mixture was poured into cooled water (15 mL) and the aqueous extracted with EtOAc (30 mL×2). The combined organic layer was washed with saturated aqueous citric acid (30 mL) and brine (20 mL). The organic layer was dried over Na₂SO₄, filtered and concentrated under reduced pressure to give the crude product. The crude product was purified by prep-HPLC (neutral conditions; column: Agela Durashell C18 150*25 5 u; mobile phase [water(10 mM NH₄CO₃)-ACN]; B %: 50%-70%, 10.5 min) to obtain isopropyl 2-(6-chloro-5,7-dimethyl-3-oxo-1,4-benzoxazin-4-yl)acetate (300 mg, 962 μmol, 40.7% yield) as an off-white solid.

¹H NMR (400 MHz, CDCl₃): δ 6.84 (s, 1H), 5.05 (sept, 1H), 4.49 (s, 2H), 4.43 (s, 2H), 2.36 (s, 3H), 2.33 (s, 3H), 1.22 (d, 6H);

LCMS: m/z (M+H)⁺=(Calculated for C₁₅H₁₈ClNO₄, 312.09) found, 312.1.

### Example 4: tert-butyl 2-(6-chloro-5,7-dimethyl-3-oxo-1,4-benzoxazin-4-yl)acetate

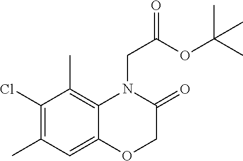 [SMILES: Cc1cc2c(c(C)c1Cl)N(CC(=O)OC(C)(C)C)C(=O)CO2]

To a solution of the compound of Intermediate 3 (500 mg, 2.36 mmol) in DMF (3.5 mL) was added NaH (189 mg, 4.72 mmol, 60% purity) at 25° C., and the resulting mixture stirred at 25° C. for 30 min. Tert-butyl 2-bromoacetate (922 mg, 4.72 mmol) was added at 25° C., and the mixture stirred at 50° C. for 12 h. The mixture was poured into cooled water (15 mL) and the aqueous extracted with ethyl acetate (30 mL×2). The combined organic layer was washed with saturated aqueous citric acid (30 mL) and brine (20 mL). The organic layer was dried over Na₂SO₄, filtered and concentrated under reduced pressure to give the crude product. The crude product was purified by prep-HPLC (neutral conditions; column: Agela Durashell C18 150*25 5 u; mobile phase [water(10 mM NH₄CO₃)-ACN]; B %: 55%-75%, 10.5 min) to obtain tert-butyl 2-(6-chloro-5,7-dimethyl-3-oxo-1,4-benzoxazin-4-yl)acetate (140 mg, 429 μmol, 18.2% yield, 99.8% purity) as a colourless oil.

¹H NMR (400 MHz, CDCl₃): δ 6.84 (s, 1H), 4.48 (s, 2H), 4.38 (s, 2H), 2.36 (s, 3H), 2.33 (s, 3H), 1.42 (s, 9H);

LCMS: m/z (M+H−C₂H₄)⁺=(Calculated for C₁₆H₂₀ClNO₄, 270.05) found, 270.1.

### Example 5: 2-(6-chloro-5,7-dimethyl-3-oxo-1,4-benzoxazin-4-yl)acetic acid

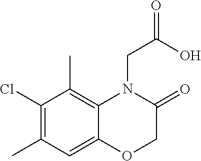 [SMILES: Cc1cc2c(c(C)c1Cl)N(CC(=O)O)C(=O)CO2]

To a solution of the compound of Example 1 (7.5 g, 26.4 mmol) in THF (40 mL) was added a solution of LiOH.H₂O (1.33 g, 31.7 mmol) in water (10 mL) at 25° C. The mixture was stirred at 25° C. for 12 h. The reaction mixture was diluted with ethyl acetate (40 mL), the organic layer was separated and the aqueous layer adjusted to pH ˜3 with HCl (6M). The aqueous was extracted with ethyl acetate (40 mL×3), and the combined organic layers dried over Na₂SO₄, filtered and concentrated under reduced pressure to provide 2-(6-chloro-5,7-dimethyl-3-oxo-1,4-benzoxazin-4-yl)acetic acid (7 g, 26.0 mmol, 98.2% yield) as an off-white solid.

Characterisation data was consistent with commercially available material.

### Example 6: 2-(6-chloro-5,7-dimethyl-3-oxo-1,4-benzoxazin-4-yl)-N,N-dimethyl-acetamide

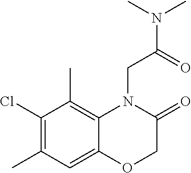 [SMILES: Cc1cc2c(c(C)c1Cl)N(CC(=O)N(C)C)C(=O)CO2]

To a solution of the compound of Example 5 (400 mg, 1.48 mmol) in dichloromethane (5 mL) was added CDI (361 mg, 2.22 mmol) and triethylamine (225 mg, 2.22 mmol) at 25° C., followed by N-methylmethanamine (145 mg, 1.78 mmol). The mixture was stirred at 25° C. for 2 h. The reaction mixture was diluted with water (10 mL) and extracted with ethyl acetate (10 mL×3). The combined organic layers were concentrated under reduced pressure to give a crude product. The crude product was purified by prep-HPLC (column: Agela Durashell C18 150*25 5 u; mobile phase: [water(10 mM NH₄HCO₃)-ACN]; B %: 25%-55%, 10.5 min) to provide 2-(6-chloro-5,7-dimethyl-3-oxo-1,4-benzoxazin-4-yl)-N,N-dimethyl-acetamide (101 mg, 340 μmol, 22.9% yield, 99.8% purity) as a white solid.

¹H NMR (400 MHz, MeOD): δ 6.89 (s, 1H), 4.63 (s, 2H), 4.49 (s, 2H), 3.10 (s, 3H), 2.97 (s, 3H), 2.39 (s, 3H), 2.32 (s, 3H);

LCMS: m/z (M+H)⁺=(Calculated for C₁₄H₁₇ClN₂O₃, 297.09) found, 297.1

### Example 7: 2-(6-chloro-5,7-dimethyl-3-oxo-1,4-benzoxazin-4-yl)-N,N-dimethyl-acetamide

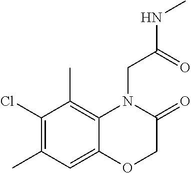 [SMILES: CNC(=O)CN1C(=O)COc2cc(C)c(Cl)c(C)c21]

To a solution of the compound of Example 5 (400 mg, 1.48 mmol) in DMF (5 mL) was added benzotriazol-1-ol (300.63 mg, 2.22 mmol)N-(3-Dimethylaminopropyl)-N′-ethylcarbodiimide hydrochloride (426.5 mg, at 25° C., followed by N-methylmethanamine (145 mg, 1.78 mmol). The mixture was stirred at 25° C. for 2 h. The reaction mixture was diluted with water (10 mL) and extracted with ethyl acetate (10 mL×3). The combined organic layers were concentrated under reduced pressure to give a crude product. The crude product was purified by prep-HPLC (column: Agela Durashell C18 150*25 5 u; mobile phase: [water(10 mM NH₄HCO₃)-ACN]; B %: 25%-55%, 10.5 min) to provide 2-(6-chloro-5,7-dimethyl-3-oxo-1,4-benzoxazin-4-yl)-N,N-dimethyl-acetamide (120 mg, 424 μmol, 28.6% yield, 99.8% purity) as a white solid.

¹H NMR (400 MHz, MeOD): δ 6.90 (s, 1H), 4.49 (s, 2H), 4.37 (s, 2H), 2.76 (s, 3H), 2.40 (s, 3H), 2.33 (s, 3H);

LCMS m/z (M+H)⁺=(Calculated for C₁₃H₁₅ClN₂O₃, 283.08) found, 283.1.

### Example 8: 2-(6-chloro-5,7-dimethyl-3-oxo-1,4-benzoxazin-4-yl)acetamide

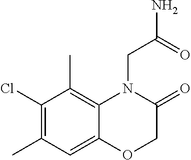 [SMILES: Cc1cc2c(c(C)c1Cl)N(CC(N)=O)C(=O)CO2]

To a solution of the compound of Example 5 (400 mg, 1.48 mmol) in dichloromethane (5 mL) was added CD (360.8 mg, 2.22 mmol) and NH₃ (4M in THF, 371 μL) at 25° C. The mixture was stirred at 25° C. for 2 h. The mixture was diluted with H₂O (10 mL) and extracted with ethyl acetate (10 mL×3). The combined organic layers were concentrated under reduced pressure to give a crude product, which was purified by prep-HPLC (column: Agela Durashell C18 150*25 5 u; mobile phase: [water(10 mM NH₄HCO₃)-ACN]; B %: 20%-55%, 10.5 min) to provide 2-(6-chloro-5,7-dimethyl-3-oxo-1,4-benzoxazin-4-yl)acetamide (150 mg, 558 μmol, 37.6% yield, 100% purity) as a white solid.

¹H NMR (400 MHz, MeOD): δ 6.89 (s, 1H), 4.49 (s, 2H), 4.41 (s, 2H), 2.42 (s, 3H), 2.32 (s, 3H);

LCMS: m/z (M+H)+=(Calculated for C₁₂H₁₃ClN₂O₃, 269.06) found, 269.1.

### Intermediate 4: 2-(6-chloro-5,7-dimethyl-3-oxo-1,4-benzoxazin-4-yl)acetonitrile

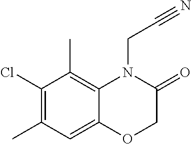 [SMILES: Cc1cc2c(c(C)c1Cl)N(CC#N)C(=O)CO2]

To a solution of the compound of Intermediate 3 (500 mg, 2.36 mmol) in DMF (7 mL) was added K₂CO₃ (816.3 mg, 5.91 mmol) followed by 2-bromoacetonitrile (425.1 mg, 3.54 mmol) at 25° C. The mixture was stirred at 25° C. for 12 h. The reaction mixture was poured into cooled water (7.5 mL), and the aqueous was extracted with ethyl acetate (15 mL*2). The combined organic layer was washed with saturated aqueous citric acid (15 mL) and brine (10 mL), then dried over Na₂SO₄, filtered and concentrated under reduced pressure. The crude product was purified by column chromatography (SiO₂, petroleum ether:ethyl acetate=20:1 to 2:1) to yield 2-(6-chloro-5,7-dimethyl-3-oxo-1,4-benzoxazin-4-yl)acetonitrile (400 mg, 1.60 mmol, 67.5% yield) as a white solid.

¹H NMR (400 MHz, CDCl₃): δ 6.87 (s, 1H), 4.59 (s, 2H), 4.51 (s, 2H), 2.50 (s, 3H), 2.35 (s, 3H).

### Example 9: 6-chloro-5,7-dimethyl-4-(2H-tetrazol-5-ylmethyl)-1,4-benzoxazin-3-one

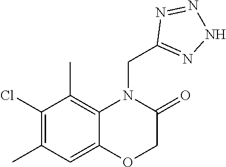 [SMILES: Cc1cc2c(c(C)c1Cl)N(Cc1nn[nH]n1)C(=O)CO2]

To a mixture of the compound of Intermediate 4 (650 mg, 2.59 mmol) in DMF (5 mL) was added NH₄Cl (832 mg, 15.6 mmol) followed by azidosodium (674 mg, 10.4 mmol) at 25° C. The resulting mixture was stirred at 100° C. for 12 h. The reaction mixture was diluted with ethyl acetate (40 mL), washed with water (30 mL) and brine (20 mL), dried over Na₂SO₄, filtered and concentrated under reduced pressure. The crude product was purified by prep-HPLC (neutral condition; column: Agela Durashell C18 150*25 5 u; mobile phase: [water(10 mM NH₄HCO₃)-ACN]; B %: 10%-40%, 10.5 min) to yield 6-chloro-5,7-dimethyl-4-(2H-tetrazol-5-ylmethyl)-1,4-benzoxazin-3-one (55 mg, 187 μmol, 7.21% yield, 99.8% purity) as a white solid.

¹H NMR (400 MHz, MeOD): δ 6.83 (s, 1H), 5.26 (s, 2H), 4.51 (s, 2H), 2.37 (s, 3H), 2.28 (s, 3H);

LCMS: m/z (M+H)+=(Calculated for C₁₂H₁₂ClN₅O₂, 294.07) found, 294.1

### Example 10: 3-(6-chloro-3-oxo-2,3-dihydro-4H-1,4-benzoxazin-4-yl)propanoic acid

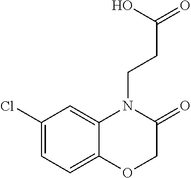 [SMILES: O=C(O)CCN1C(=O)COc2ccc(Cl)cc21]

3-(6-chloro-3-oxo-2,3-dihydro-4H-1,4-benzoxazin-4-yl)propanoic acid was purchased from Vitas-M laboratory.

### BIOLOGICAL EXAMPLES

### Biological Example 1: Expression and Purification of Recombinant P. fluorescens KMO

The codon optimised gene for P. fluorescens KMO (accession number: Q84HF5) containing mutations of two cysteine residues (252 and 461) to serine was synthesised (GeneArt, ThermoFisher) sub-cloned into pET17b and transformed into E. coli BL21 (DE3) competent cells for expression. Protein was expressed by growing transformed cells in auto induction LB medium (Fermentas) containing 100 μg/mL for 24 hours at 22° C. Cells were harvested by a centrifugation at 6000 g for 15 min at 4° C., resuspended in lysis buffer (20 mM HEPES pH 7.5, 10 mM NaCl, 1 mM DTT) supplemented with protease inhibitor cocktail and lysed by sonication (15×15 sec). The cell lysate was centrifuged at 180,000 g for 1 hour at 4° C. to remove cell debris. The soluble cell lysate was loaded onto Q-sepharose column equilibrated with anion exchange buffer (20 mM HEPES pH 7.5, 1 mM DTT with 10 mM NaCl) and bound protein was eluted with a gradient (5 column volumes) of increasing NaCl concentration from 50 to 100 mM in anion exchange buffer. Fractions containing KMO were pooled and precipitated in 50% saturated ammonium sulphate by adding precipitation buffer (20 mM HEPES pH 7.5, 3 M ammonium sulphate, 1 mM DTT). The precipitated protein was pelleted by centrifugation at 12,000 g for 20 min, resuspended in a small volume of size exclusion buffer (20 mM HEPES pH 7.0, 150 mM sodium acetate, 1 mM DTT) and passed down a Superdex 75 column. Pure PfKMO was pooled and stored at −80° C.

### Biological Example 2: Expression and Purification of Recombinant Homo sapiens KMO

The gene encoding full-length human KMO was synthesized (GeneScript) and codon optimized for overexpression in mammalian cells. The gene was subcloned into the baculovirus transfer vector pAcGHLT-A-glutathione S-transferase as a Ndel-EcoRI fragment and transfected into Hi5 cells along with linearized baculovirus using the following sense and antisense primers:

5′-GGCATATGCATGGACAGCAGCGTGATCCAGCGGAAG-3′;
5′-CCCGAATTCCTACCGGCTGATCAGGTTGCTG-3′.

Hi5 cells (1.5×106) were infected with recombinant virus for 72 h at 28° C. A WAVE

Bioreactor System (GE Healthcare Life Sciences) was used to grow batches of 5-litres of cell culture. Cultured cells were lysed in 20 mM potassium phosphate buffer, pH 7.5, 10% glycerol, 0.5% n-dodecyl β-D-maltoside (DDM), 150 mM NaCl, 7 mM 2-mercaptoethanol and 50 μM FAD supplemented with protease inhibitors (Sigma-Aldrich). Soluble lysate was incubated with 3 ml pre-equilibrated glutathione uniflow resin (Clontech). The resin was then packed and washed with buffer B (20 mM potassium phosphate buffer, pH 7.5, 10% glycerol, 0.012% DDM, 150 mM NaCl, 7 mM 2-mercaptoethanol, 50 μM FAD). Fractions (0.5 ml) were eluted with buffer C (buffer B+33 mM glutathione) and fractions containing KMO pooled, concentrated and loaded onto a Superdex 200 (10/30) size-exclusion chromatography column. Pure KMO was pooled and stored at −80° C.

### Biological Example 3: Inhibition of Recombinant P. fluorescens KMO

Small molecule inhibition of the recombinant P. fluorescens kynurenine 3-monooxygenase (PIKMO) of Biological Example 1 was examined. Determination of IC₅₀ values for each compound were carried out using 0.1 μM PfKMO at fixed concentrations of L-Kyn (250 μM) and NADPH (200 μM) in reaction buffer (20 mM HEPES, 10 mM sodium acetate, pH 7.5, 2 mM DTT). KMO activity was measured in the presence of a range of compound concentrations by monitoring the consumption of NADPH at 340 nm upon addition of enzyme via a Cary 50 UV-Vis spectrophotometer (Agilent Technologies). All measurements were performed in triplicate. The IC₅₀ values for each compound were obtained by plotting the percentage inhibition versus the inhibitor concentration and fitting to the Morrison equation using Origin 9.0 software (OriginLab, Northampton, Mass.).

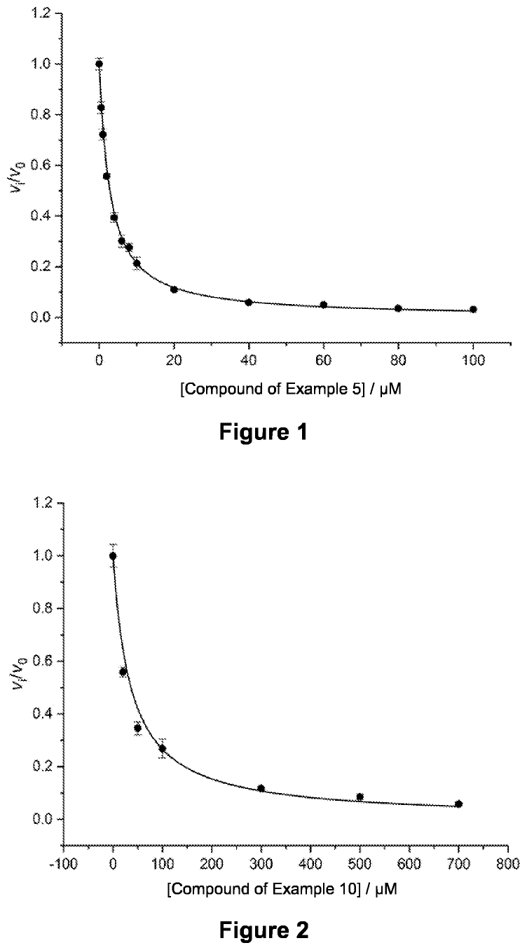

Results obtained for the compounds of Examples 1 to 10 are shown in the following table. Results obtained for the compounds of Examples 5 and 10 are also represented in FIGS. 1 and 2, respectively.

| Compound Example | IC₅₀ for PfKMO inhibition (μM) |
|---|---|
| 5 | 9.31 |
| 1 | >100 |
| 2 | >100 |
| 3 | >100 |
| 4 | >100 |
| 6 | >100 |
| 7 | >100 |
| 8 | >100 |
| 9 | 16.96 |
| 10 | 44.4 |

### Biological Example 4: Inhibition of Recombinant Homo sapiens KMO

Small molecule inhibition of the recombinant Homo Sapiens kynurenine 3-monooxygenase (HsKMO) of Biological Example 2 was examined. Determination of IC₅₀ values for each compound were carried out using 0.5 μM HsKMO at fixed concentrations of L-Kyn (250 μM) and NADPH (200 μM) in reaction buffer (20 mM potassium phosphate pH 8.0, 2 mM 2-mercaptoethanol). KMO activity was measured in the presence of a range of compound concentrations by monitoring the consumption of NADPH at 340 nm upon addition of enzyme via a Cary 50 UV-Vis spectrophotometer (Agilent Technologies). All measurements were performed in triplicate. The IC₅₀ values for each compound were obtained by plotting the percentage inhibition versus the inhibitor concentration and fitting to the Morrison equation using Origin 9.0 software (OriginLab, Northampton, Mass.).

Results obtained for the compounds of Examples 1 to 10 are shown in the following table.

| Compound Example | IC₅₀ for HsKMO inhibition (μM) |
|---|---|
| 5 | 2.60 |
| 1 | >100 |
| 2 | >100 |
| 3 | >100 |
| 4 | >100 |
| 6 | >100 |
| 7 | >100 |
| 8 | >100 |
| 9 | 1.28 |
| 10 | 36.7 |

### Biological Example 5

The mechanism of inhibition of PfKMO by the compound of Example 5 was investigated by carrying out a full steady-state kinetic analysis at varying concentrations of the compound (0, 5, 10, 20 and 30 μm) and the two substrates, L-Kyn and NADPH. The studies were carried out by measuring reaction rates at a saturating concentration (250 μM) of one substrate and varying concentrations (5-200 μM NADPH; 10-250 μM L-Kyn) of the other substrate.

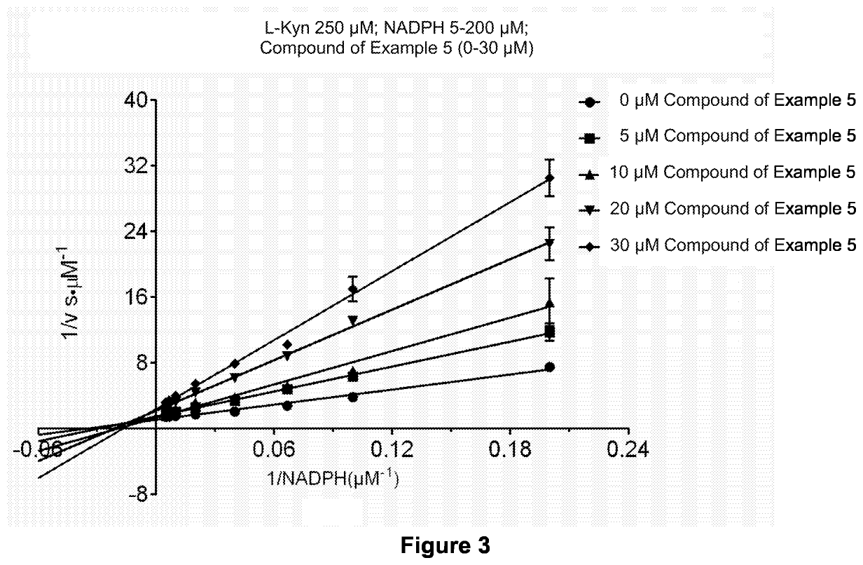

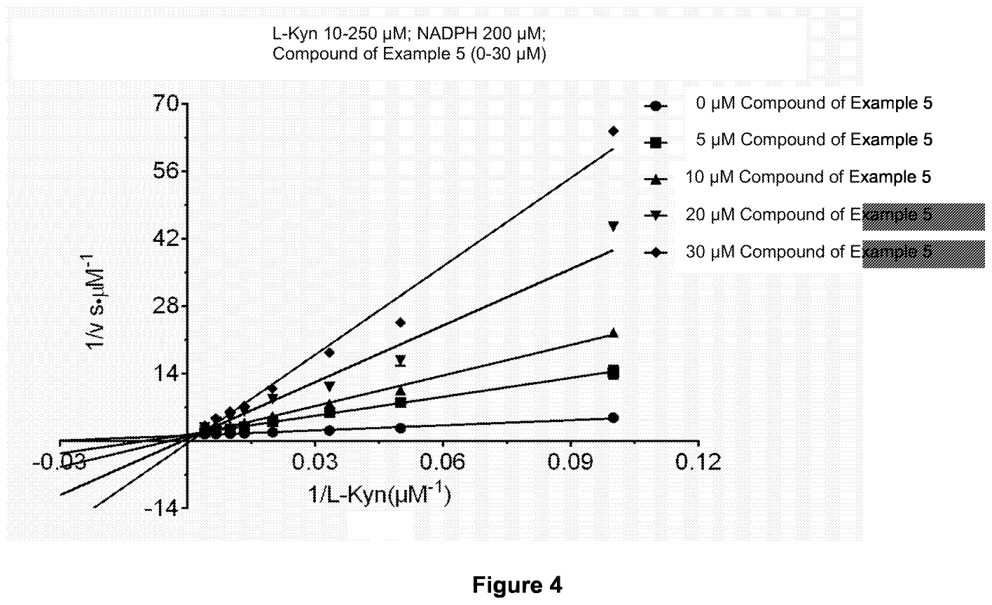

Results obtained for the compound of Example 5 are represented in the Lineweaver-Burke plots shown in FIGS. 3 and 4.

### Biological Example 6: Mouse Brain Penetration

The compounds of Examples 1 to 3 and 5 were dosed as a bolus solution intravenously at 1 mg free base/kg to female C57BL6 mice (six mice per compound). For the compound of Example 5 the dose volume was 5 mL/kg and the dose vehicle was 5 vol. % DMSO; 95 vol. % saline. For the compounds of Examples 1 to 3, the dose volume was 5 mL/kg and the dose vehicle was 5 vol. % DMSO; 40 vol. % PEG400; 55 vol. % MilliQ H₂O.

At 5 minutes and 60 minutes following intravenous bolus injection of test compound, mice (three mice per compound per timepoint) were placed under terminal anaesthesia with isofluorane. A blood sample was taken by cardiac puncture, the mice decapitated and the brain removed. Brain weight was noted and each brain homogenised with two volumes of methanol (2 mL/g brain). Each blood sample was diluted with distilled water (1:10). Diluted blood and the brain homogenates were then stored frozen until preparation and analysis by UPLC-MS/MS.

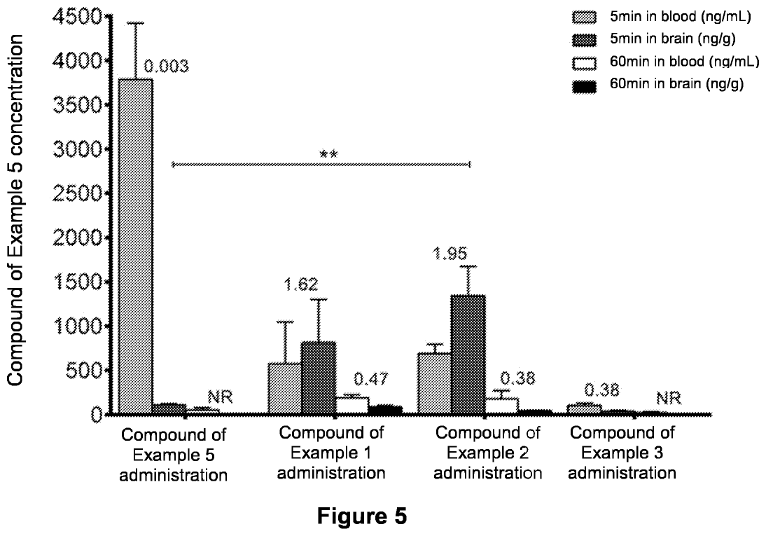

For each mouse at each timepoint for the compound of Example 1 to 3 and 5 the concentration in blood (ng/mL) and brain (ng/g) of the compound of Example 5 was reported. The mean value obtained for each timepoint is shown in FIG. 5.

These data demonstrate that the compounds of Examples 1 to 3 are metabolised to produce the compound of Example 5 in vivo.

1. A compound of formula I 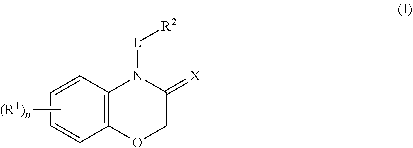
or a pharmaceutically-acceptable salt thereof, wherein:
L represents C₁₋₆ alkyl optionally substituted by one or more fluoro;
n represents 2 to 4;
each R¹ independently represents C₁₋₆ alkyl group optionally substituted by one or more groups independently selected from G^{1a} or a halo group, wherein at least one R¹ is a C₁₋₆ alkyl group optionally substituted by one or more groups independently selected from G^{1a};
each G^{1a} independently represents halo, —CN, —N(R^{a3})R^{b3}, —OR^{c3}, —SR^{d3} or ═O;
each R^{a3}, R^{b3}, R^{c3} and R^{d3} independently represents H or C₁₋₆ alkyl optionally substituted by one or more fluoro;
or R^{a3} and R^{b3} are linked together to form, along with the nitrogen atom to which they are attached, a 3- to 6-membered ring, which ring optionally contains one further heteroatom and which ring optionally is substituted by one or more groups independently selected from fluoro, C₁₋₃ alkyl optionally substituted by one or more fluoro, and ═O;
R² represents C(O)OR^{a2}, C(O)NR^{b2}R^{c2}, or tetrazole;
each R^{a2}, R^{b2} and R^{c2} independently represents H, C₁₋₆ alkyl, phenyl, or CH₂-phenyl, wherein each of the latter three groups may be optionally substituted by one or more fluoro; and
X represents O or S,
with the proviso that the compound of formula I is not 2-(6-chloro-5,7-dimethyl-3-oxo-1,4-benzoxazin-4-yl) acetic acid.

2. A compound as claimed in claim 1, wherein at least two R¹ groups are a C₁₋₆ alkyl group optionally substituted by one or more groups independently selected from G^{1a}.

3. A compound according to claim 1, wherein two R¹ groups are methyl.

4. A compound according to claim 1, wherein at least one R¹ group is Cl.

5. A compound according to claim 1, wherein each R^{a2}, R^{b2} and R^{c2} independently represents H or C₁₋₃ alkyl optionally substituted by one or more fluoro.

6. A compound according to claim 1, wherein X represents O.

7. A compound according to claim 1, wherein L represents C₁₋₃ alkyl optionally substituted by one or more fluoro.

8. A compound according to claim 7, wherein L represents C₁ alkyl (i.e. methyl) optionally substituted by one or more fluoro.

9. A compound according to claim 1, wherein the compound is: 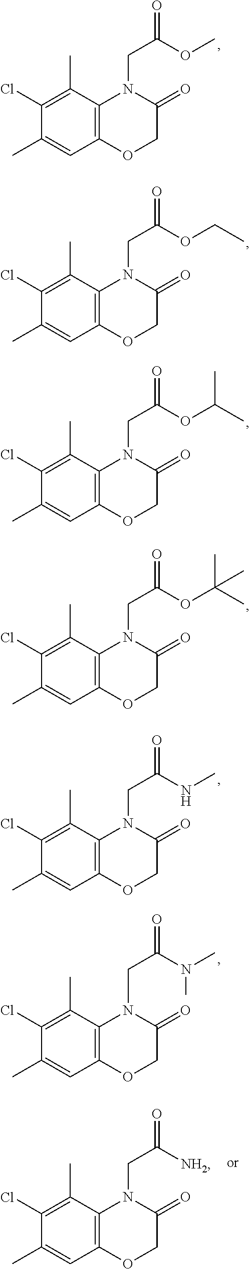 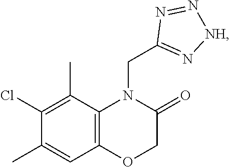 [SMILES: Cc1cc2c(c(C)c1Cl)N(Cc1nn[nH]n1)C(=O)CO2]
or a pharmaceutically-acceptable salt thereof.

10. A compound as defined in claim 1, or 2-(6-chloro-5,7-dimethyl-3-oxo-1,4-benzoxazin-4-yl)acetic acid or 3-(6-chloro-3-oxo-2,3-dihydro-4H-1,4-benzoxazin-4-yl)propanoic acid for use as a pharmaceutical.

11. (canceled)

12. A method of treating a neurodegenerative or neuroinflammatory disease comprising administering to a patient in need thereof a therapeutically effective amount of a compound as defined in any one of claim 1 to 9, or 2-(6-chloro-5,7-dimethyl-3-oxo-1,4-benzoxazin-4-yl)acetic acid or 3-(6-chloro-3-oxo-2,3-dihydro-4H-1,4-benzoxazin-4-yl)propanoic acid.

13. (canceled)

14. The method of claim 12, wherein the neurodegenerative or neuroinflammatory disease is selected from the group consisting of: Alzheimer's disease, Parkinson's disease, amyotrophic lateral sclerosis and Huntington's disease.

15. The method of claim 12, with the proviso that the compound of formula I is not 2-(6-chloro-5,7-dimethyl-3-oxo-1,4-benzoxazin-4-yl) acetic acid.

16. A pharmaceutical composition comprising a compound as defined in claim 1, or 2-(6-chloro-5,7-dimethyl-3-oxo-1,4-benzoxazin-4-yl)acetic acid or 3-(6-chloro-3-oxo-2,3-dihydro-4H-1,4-benzoxazin-4-yl)propanoic acid, and optionally one or more pharmaceutically-acceptable adjuvant, diluent and/or carrier.

17. A combination product comprising:
(A) a compound as defined in claim 1, or 2-(6-chloro-5,7-dimethyl-3-oxo-1,4-benzoxazin-4-yl)acetic acid or 3-(6-chloro-3-oxo-2,3-dihydro-4H-1,4-benzoxazin-4-yl)propanoic acid; and
(B) one or more other therapeutic agent that is useful in the treatment of a neurodegenerative or neuroinflammatory disease,
wherein each of component (A) and (B) is formulated in admixture, optionally with one or more pharmaceutically-acceptable adjuvant, diluent or carrier.

18. A kit-of-parts comprising:
(a) a pharmaceutical composition as defined in claim 1; and
(b) one or more other therapeutic agent that is useful in the treatment of a neurodegenerative or neuroinflammatory disease, optionally in admixture with one or more pharmaceutically-acceptable adjuvant, diluent or carrier,
which components (a) and (b) are each provided in a form that is suitable for administration in conjunction with the other.

19. A process for the preparation of a compound as defined in claim 1, which process comprises:
(i) reaction of a compound of formula II 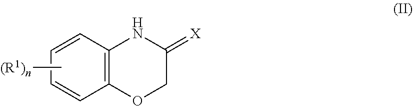
wherein R¹, n and X are as defined in any one of claims 1 to 9, with a compound of formula III 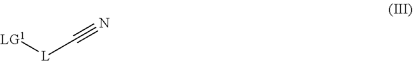
wherein L is as defined in any one of claims 1 to 9 and LG¹ represents a suitable leaving group, in the presence of a suitable base and in the presence of a suitable solvent, under conditions known to those skilled in the art, followed by reaction of the resultant compound of formula IV 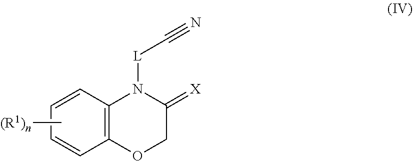
wherein R¹, n, X and L are as defined in any one of claims 1 to 9, with an azide in the presence of a suitable co-reagent and in the presence of a suitable solvent, under conditions known to those skilled in the art;
(ii) reaction of a compound of formula II 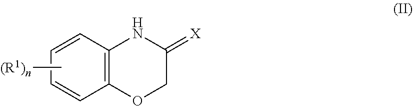
wherein R¹ and X are as defined in any one of claims 1 to 9, with a compound of formula V 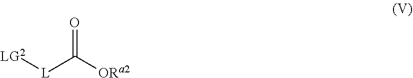
wherein L and R^{a2} are as defined in any one of claims 1 to 9 and LG² represents a suitable leaving group, in the presence of a suitable base and in the presence of a suitable solvent, under conditions known to those skilled in the art;
(iii) reaction of a compound of formula IA 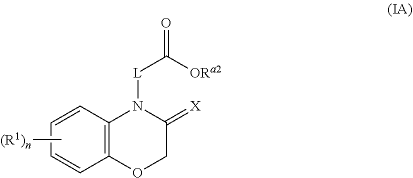
wherein R¹, n, R^{a2}, X and L are as defined in any one of claims 1 to 9, with a suitable aqueous base in the presence of a suitable solvent, under conditions known to those skilled in the art; or
(iv) reaction of a compound of formula IB 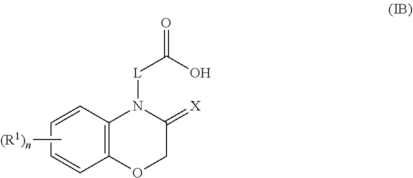
wherein R¹, n, X and L are as in any one of claims 1 to 9, with a compound of formula VI 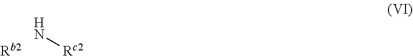
wherein R^{b2} and R^{c2} are as defined in any one of claims 1 to 9, in the presence of a suitable coupling reagent and in the presence of a suitable solvent, optionally in the presence of a suitable base.

20. The method of claim 14, with the proviso that the compound of formula I is not 2-(6-chloro-5,7-dimethyl-3-oxo-1,4-benzoxazin-4-yl) acetic acid.


SEQUENCE LISTING (2 sequences)

SEQ ID NO: 1 - DNA, 36 bp, Artificial Sequence
  Primer
ggcatatgcatggacagcagcgtgatccagcggaag

SEQ ID NO: 2 - DNA, 31 bp, Artificial Sequence
  Primer
cccgaattcctaccggctgatcaggttgctg